(19) United States
(12) Patent Application Publication
(10) Pub. No.: US 2021/0139895 A1
(43) Pub. Date: May 13, 2021

(54) PHARMACEUTICAL COMPOSITION FOR PREVENTING OR TREATING CANCER

(71) Applicant: PROSTEMICS CO., LTD., Seoul (KR)

(72) Inventors: Eun Wook Choi, Seoul (KR); Byung Soon Park, Seoul (KR); Won Jong Lee, Seoul (KR); Dohyung Kim, Incheon (KR); Jun Won Lee, Seoul (KR)

(21) Appl. No.: 16/623,249

(22) PCT Filed: Jun. 18, 2018
(86) PCT No.: PCT/KR2018/006865

§ 371 Date: Dec. 16, 2019

(30) Foreign Application Priority Data
Jun. 16, 2017 (KR) 10-2017-0076793

(51) Int. Cl. C12N 15/113; A61P 35/04

(52) U.S. Cl. CPC C12N 15/113; A61P 35/04; C12N 2310/321; C12N 2310/315; C12N 2310/141

(57) ABSTRACT

The present invention relates to a pharmaceutical composition for preventing or treating cancer, comprising, as an active ingredient, miRNA comprising a seed sequence represented by the nucleotide sequence of any one of SEQ ID NOs: 1 to 3; an expression vector comprising the miRNA; or a transformant transformed with the expression vector. The miRNA provided by the present invention can effectively inhibit growth of cancer cells as well as cancer stem cells so that cancer is prevented and/or treated; and, furthermore, the miRNA can also prevent drug resistance, metastasis, and recurrence of cancer.

### TECHNICAL FIELD

The present invention relates to a pharmaceutical composition capable of effectively preventing or treating cancer.

### BACKGROUND ART

MicroRNAs (miRNAs) are small, non-coding RNAs consisting of 18 to 25 nucleotides (nt) in length, which regulate gene expression by binding to the 3′-untranslated region (UTR) of their target genes (Bartel D P, et al., Cell 116: 281-297, 2004; Lewis B P, et al., Cell 120: 15-20, 2005) and which are processed from introns, exons or intergenic regions (Rodriguez A, et al., Genome Res 14: 1902-1910, 2004). First, miRNAs are transcribed by RNA polymerase into primary miRNA (pri-miRNA) molecules that contain several thousand nucleotides. The pri-miRNAs are then sequentially processed by a microprocessor [Drosha RNase endonuclease and DiGeorge syndrome region gene 8 protein (DGCR8)], to form approximately 70 nt-stem-loop intermediates known as miRNA precursors (Lee Y, et al., EMBO J 21: 4663-4670, 2000; Zeng Y, et al., Proc Natl Acad Sci USA 100: 9779-9784, 2003). The pre-miRNAs are then exported from the nucleus into the cytoplasm via Exportin-5 (EXP5), with its cofactor Ran-GTP, where these pre-miRNAs are processed into 18 to 25 nt mature miRNA duplexes by RNase endonuclease Dicer (Lee Y, et al., EMBO J 23: 4051-4060, 2004; Shenouda S K, et al., Cancer Metastasis Rev 28: 369-378, 2009). Along with an Argonaute protein, the mature miRNA duplex as single-stranded RNA is integrated into an RNA-induced silencing complex, which induces either cleavage or translational inhibition of targeted mRNA (Diederichs S, et al., Cell 131: 1097-1108, 2007; Hammond S M, et al., Nature 404: 293-296, 2000; Martinez et al., Cell 110: 563-574, 2002). miRNAs are implicated in a variety of biological processes associated with cancer development, including proliferation and invasion of cancer cells, and miRNA expression is bidirectionally regulated in many types of cancers (Esquela-Kerscher A, et al., Nat Rev Cancer 6: 259-269, 2006).

Cancer is one of the most common causes of death worldwide. About 10 million new cancer cases occur each year; and cancer accounts for about 12% of all deaths, making it the third leading cause of death.

Among cancers, breast cancer is the most common malignancy in women which causes more than 40,000 deaths every year. For this cancer, early diagnosis is very important; however, despite many known anti-cancer therapies, there has been no improvement in survival rate in a case where cancer is highly advanced or metastasized.

Chemotherapy, a representative anti-cancer therapy, is currently used as the most efficient therapy for treating cancer, either alone or in combination with other therapies such as radiotherapy. However, although efficacy of cancer therapeutic drugs in chemotherapy varies depending on their ability to kill cancer cells, there is a problem in that the drugs may act on normal cells as well as cancer cells when the drugs are used.

A hypothesis that cancer stem cells are cancer cells having unlimited regenerative capacity and tumors originate from such stem cells was confirmed with study results published in the late 1990s, the results showing that transplantation of a group of cells, which can become cancer stem cells in acute myelogenous leukemia, into immunosuppressed rats reproduces human leukemia in the rats. Since then, cancer stem cells have been proven to exist in breast cancer, which confirms existence of stem cells in solid carcinomas.

Various heterogeneities of malignancies coincide with various differentiation characteristics of stem cells; and drug resistance of cancer cells, which is constantly expressed despite many targeted therapies, coincides with basic characteristics of stem cells. As a result, it is possible to associate tumor development with stem cells, and thus cancer stem cells may become a new field of targeted therapy.

Several therapeutic methods have been designed based on the cancer stem cell hypothesis. Among these, the best-known method is a method in which the self-renewal pathway of cancer stem cells is used. In such a therapy, it is important that only the self-renewal of cancer stem cells should be targeted while maintaining the self-renewal of normal stem cells. For example, Notch signaling proceeds by means of an enzyme called gamma secretase. In this regard, in a case where an inhibitor (gamma secretase inhibitor) against the enzyme is used for breast cancer in which Notch1 is overexpressed, it is possible to achieve a tumor inhibitory effect. There is a recent report that an anti-cancer effect is observed even in a case where the hedgehog signaling system is targeted. The report has shown that dramatic tumor contraction occurs when tumor xenograft animal models receive the hedgehog inhibitor, cyclopamine. In addition, it is known that PI3K/AKT, MAPK, and JAK2/STAT3 signaling pathways are associated with cancer stem cells.

However, research on cancer stem cells has many limitations so far, and their roles in tumor formation or maintenance have not yet been clearly identified. In order to efficiently achieve treatment that targets only cancer stem cells without causing damage to normal stem cells, knowledge and understanding on molecular biological characteristics important for maintenance and regulation of cancer stem cells or regulatory pathways thereof are required.

To date, there is little research on anti-cancer agents or natural product-derived extracts which directly target cancer stem cells. In the prior art, research has been conducted to inhibit cancer stem cells, in experiments in which direct target genes of the cancer stem cells are inhibited, or by inhibiting upstream signaling proteins in the cancer stem cells. However, in many tumor patients, such targeting experiments have difficulties due to mutations in oncogenes or proteins.

Thus, improvement in selectivity of anti-cancer drugs for cancer stem cells will increase efficacy of chemotherapy with such anti-cancer drugs, thereby clearly allowing the drugs to be used at lower doses. Therefore, there is a need for an improved approach that can selectively inhibit growth of cancer stem cells for treatment and prevention of cancer.

### Technical Problem

An object of the present invention is to provide a pharmaceutical composition capable of preventing and/or treating cancer.

Another object of the present invention is to provide a pharmaceutical composition capable of effectively inhibiting growth of cancer stem cells so that not only cancer is prevented and/or treated, but also drug resistance, metastasis, and recurrence of cancer are prevented.

Other objects and advantages of the present invention will become more apparent from the following detailed description, claims, and drawings.

### Solution to Problem

The present inventors have found that hsa-miR-328-3p, hsa-miR-6514-5p, and hsa-miR-503-3p mRNAs not only effectively inhibit growth and proliferation of cancer stem cells as well as cancer cells, but also inhibit metastasis of cancer, thereby reaching the present invention.

According to an embodiment of the present invention, there is provided a pharmaceutical composition for preventing or treating cancer, comprising, as an active ingredient, miRNA comprising a seed sequence represented by the nucleotide sequence of any one of SEQ ID NOs: 1 to 3; an expression vector comprising the miRNA; or a transformant transformed with the expression vector.

According to another embodiment of the present invention, there is provided a pharmaceutical composition for inhibiting growth of cancer stem cells, comprising, as an active ingredient, miRNA comprising a seed sequence represented by the nucleotide sequence of any one of SEQ ID NOs: 1 to 3; an expression vector comprising the miRNA; or a transformant transformed with the expression vector.

According to yet another embodiment of the present invention, there is provided a pharmaceutical composition for preventing or treating metastasis of cancer, comprising, as an active ingredient, miRNA comprising a seed sequence represented by the nucleotide sequence of any one of SEQ ID NOs: 1 to 3; an expression vector comprising the miRNA; or a transformant transformed with the expression vector.

According to still yet another embodiment of the present invention, there is provided a method for preventing or treating cancer, comprising a step of administering, to a subject in need of treatment, an effective amount of miRNA comprising a seed sequence represented by the nucleotide sequence of any one of SEQ ID NOs: 1 to 3; an expression vector comprising the miRNA; or a transformant transformed with the expression vector, so that cancer is prevented or treated.

According to still yet another embodiment of the present invention, there is provided a method for inhibiting growth of cancer stem cells, comprising a step of administering, to a subject in need of treatment, an effective amount of miRNA comprising a seed sequence represented by the nucleotide sequence of any one of SEQ ID NOs: 1 to 3; an expression vector comprising the miRNA; or a transformant transformed with the expression vector, so that growth of cancer stem cells is inhibited.

According to still yet another embodiment of the present invention, there is provided a method for preventing or treating metastasis of cancer, comprising a step of administering, to a subject in need of treatment, an effective amount of miRNA comprising a seed sequence represented by the nucleotide sequence of any one of SEQ ID NOs: 1 to 3; an expression vector comprising the miRNA; or a transformant transformed with the expression vector, so that metastasis of cancer is prevented or treated.

In the present invention, the nucleotide sequence represented by SEQ ID NO: 1 may be a seed sequence of hsa-miR-328-3p miRNA represented by the nucleotide sequence of SEQ ID NO: 4 (CUGGCCCUCUCUGCCCUUCCGU).

In the present invention, the nucleotide sequence represented by SEQ ID NO: 2 may be a seed sequence of hsa-miR-6514-5p miRNA represented by the nucleotide sequence of SEQ ID NO: 5 (UAUGGAGUGGACUUUCAGCUGGC).

In the present invention, the nucleotide sequence represented by SEQ ID NO: 3 may be a seed sequence of hsa-miR-503-3p miRNA represented by the nucleotide sequence of SEQ ID NO: 6 (GGGGUAUUGUUUCCGCUGCCAGG).

In the present invention, the nucleotide sequence represented by SEQ ID NO: 4 may be a sequence of hsa-miR-328-3p miRNA.

In the present invention, the nucleotide sequence represented by SEQ ID NO: 5 may be a sequence of hsa-miR-6514-5p miRNA.

In the present invention, the nucleotide sequence represented by SEQ ID NO: 6 may be a sequence of hsa-miR-503-3p miRNA.

In the present invention, the “seed sequence” refers to a nucleotide sequence of a partial region in miRNA by which the miRNA binds, with perfect complementarity, to its target when it recognizes the target, the nucleotide sequence being a part that is essentially required for miRNA to bind to its target and also a site that performs a substantially effective function.

Therefore, in the present invention, any miRNA may be included, without limitation, in the miRNA as long as it is the polynucleotide represented by any one of SEQ ID NOs: 1 to 3, or miRNA comprising the polynucleotide. Preferably, the miRNA is one in which a nucleic acid molecule that functions to treat cancer or cancer stem cells is present contiguously to the polynucleotide represented by any one of SEQ ID NO: 1 to 3, and may be in the form of mature miRNA in which the nucleic acid molecule has a total length of 14 to 29 nt, more preferably 19 to 25 nt, and more preferably 21, 22, or 23 nt.

More preferably, in the present invention, the miRNA may include at least one of the following: (1) at least one miRNA selected from the group consisting of hsa-miR-328-3p miRNA, hsa-miR-6514-5p miRNA, and hsa-miR-503-3p miRNA; and (2) miRNA comprising the nucleotide sequence represented by any one of SEQ ID NOs: 1 to 3, the miRNA consisting of a nucleotide sequence that has at least 80%, at least 90%, or at least 95% homology with the nucleotide sequence of (1).

As described above, miRNAs used in the present invention are in a concept which is intended to include functional equivalents to a nucleic acid molecule constituting the miRNA, for example, variants that is obtained by modifying some nucleotide sequences of the miRNA nucleic acid molecule through deletion, substitution, or insertion and plays a functionally equivalent action to the miRNA nucleic acid molecule despite such modification. For example, the miRNAs of the present invention may exhibit at least 80% homology with the nucleotide sequence of each corresponding SEQ ID NO; and the miRNAs may include those exhibiting preferably 90% homology therewith, and more preferably at least 95% homology therewith. Such homology may be readily determined by comparing a nucleotide sequence with a corresponding portion of its target gene using computer algorithms well known in the art, for example, Align or BLAST algorithm.

In addition, in the present invention, the miRNA may be mature miRNA as described above, and may be a miRNA precursor, primary miRNA (pri-miRNA), or a miRNA precursor in the form of plasmid. However, in the present invention, the nucleic acid molecule constituting the miRNA precursor or primary miRNA may have a length of 50 to 150 nt, preferably 50 to 100 nt, and more preferably 65 to 95 nt.

In addition, in the present invention, the miRNA may exist in a single-stranded or double-stranded form. While mature miRNA molecules exist predominantly in a single-stranded form, precursor miRNA molecules may contain a partial self-complementary structure (for example, stem-loop structure) that may form a double strand.

In addition, at least one of the nucleic acid molecules constituting the miRNAs of the present invention may be in a form that contains a phosphorothioate structure, the phosphorothioate structure being obtained by substitution of the phosphate backbone structure with elemental sulfur; a form obtained by substitution with a DNA, peptide nucleic acid (PNA), or locked nucleic acid (LNA) molecule; or a form obtained by substitution of the sugar 2′ hydroxyl group with a methylated, methoxylated, or fluorinated structure.

The miRNA of the present invention may be isolated or prepared using standard molecular biology techniques, for example, chemical synthesis or recombinant methods, or may be commercially available.

In the present invention, as described above, the miRNA nucleic acid molecule may be provided in the form of being contained in an expression vector.

In the present invention, the expression vector is preferably a non-viral vector or a viral vector. The non-viral vector is preferably plasmid DNA. For the viral vector, lentivirus, retrovirus, adenovirus, herpes virus, and avipox virus vectors, and the like may be used. However, the present invention is not limited thereto.

In addition, in the present invention, the expression vector preferably further contains a selection marker in order to facilitate selection of transformed cells. Examples of the marker may include those conferring selectable phenotypes such as drug resistance, auxotrophy, resistance to cytotoxic agent or surface protein expression, for example, green fluorescent protein, puromycin, neomycin, hygromycin, histidinol dehydrogenase (hisD), guanine phosphoribosyltransferase (Gpt), and the like.

In the present invention, the expression vector may be introduced into a host cell to provide a transformed transformant.

In the present invention, the host cell is preferably a somatic cell of a mammal, including a human, and more preferably, a cell at tissue site intended for human treatment, or a cancer cell or a cancer stem cell at such site; however, the host cell is not limited thereto.

In addition, in the present invention, regarding a method for introducing the expression vector into a host cell, the expression vector may be introduced into the cell together with a delivery reagent including G-fectin, Mirus TrasIT-TKO lipophilic reagent, lipofectin, lipofectamine, cellfectin, cationic phospholipid nanoparticles, a cationic polymer, cationic micelle, cationic emulsion, or liposome, or intracellular uptake of the expression vector may be increased by conjugation with a biocompatible polymer such as polyethylene glycol; however, the method is not limited thereto.

The miRNA, expression vector, or transformant provided by the present invention may inhibit growth or proliferation of cancer cells or cancer stem cells, or induce death thereof; or may inhibit stemness of cancer stem cells, specifically, inhibit expression of at least one of NANOG and OCT4, which are stemness-related markers in cancer stem cells, or increase expression of at least one of CK18 and KRT20 whose expression is inhibited in cancer stem cells, so that loss of stemness is induced.

In the present invention, the “subject in need of treatment” may be a subject who has or is suspected of having symptoms of cancer or cancer metastasis, and thus is in need of preventing, ameliorating, or treating cancer or cancer metastasis by inhibiting growth or proliferation of cancer or cancer stem cells, or the like.

In general, “cancer stem cell” refers to a cancer cell in a comprehensive sense, which has self-renewal or differentiation capacity that is characteristic of stem cells.

The “cancer” typically refers to or indicates a physiological condition characterized by unregulated cell growth in mammals. Cancer to be treated and prevented in the present invention may be, depending on the development site thereof, breast cancer, colorectal cancer, uterine cancer, fallopian tube cancer, ovarian cancer, gastric cancer, brain cancer, head and neck cancer, rectal cancer, small intestine cancer, esophageal cancer, lymph gland cancer, gallbladder cancer, lung cancer, skin cancer (or melanoma), kidney cancer, bladder cancer, blood cancer, pancreatic cancer, prostate cancer, thyroid cancer, endocrine gland cancer, oral cancer, liver cancer, or the like, with breast cancer, colorectal cancer, uterine cancer, fallopian tube cancer, ovary cancer, gastric cancer, brain cancer, head and neck cancer, rectal cancer, small intestine cancer, esophageal cancer, lymph gland cancer, gallbladder cancer, lung cancer, skin cancer (or melanoma), kidney cancer, bladder cancer, blood cancer, pancreatic cancer, prostate cancer, thyroid cancer, endocrine gland cancer, oral cancer or liver cancer being preferred, and breast cancer, melanoma, lung cancer, blood cancer, or colorectal being more preferred; however, cancer is not limited thereto as long as cancer progression, such as tumor differentiation and/or proliferation, belongs to a type of cancer that is dependent on the cancer stem cells described in the present invention.

It has been reported that such cancer stem cells capable of differentiating into cancer cells are present in a proportion of about 1 to 2% in the malignant tumor tissue, and that although the cancer stem cells have self-renewal capacity that is characteristic of normal stem cells and pluripotency that can differentiate into other cells, due to abnormality in self-regulatory function, these cells increase their cell number by activation of cell division and differentiate themselves into malignant tumor cells.

Since the revelation of existence of cancer stem cells in leukemia in 1997 (Blood, 1997), evidence has also been shown that cancer stem cells exist in breast cancer (PNAS, 2003), brain tumor (Nature, 2004), prostate cancer (Cancer Res, 2005), colorectal cancer (Nature, 2007), and melanoma (Nature, 2008). A small number of cancer stem cells contained in tumors have emerged as the main cause of malignant degeneration, anti-cancer resistance, and recurrence of tumor.

Cancer stem cells have markers that are discriminated from other cancer cells. As the cancer stem cell markers, various carcinoma-specific cancer stem cell markers as shown in Table 1 below are known.

TABLE 1

| Carcinoma | Cancer stem cell marker | Source |
|---|---|---|
| Glioblastoma | CD133 | |
| Kidney cancer | CD105, CD133 | Contemp Oncol (Pozn). |
| | | 2015; 19(1A): A44-A51 |
| Thyroid cancer | ABCG2, MRP1, LRP, | J Clin Pathol. 2014 |
| | and CXCR4 | February; 67(2): 125-33 |
| Acute myelogenous | CD34+/CD38− | |
| leukemia (AMM) | | |
| Multiple myeloma | CD133− | |
| Breast cancer | CD44+/CD24−/low | Breast Cancer Res. 2007; |
| | | 9(3): 303 |
| Colorectal cancer | CD133+ | |
| Prostate cancer | CD44+/α2β1hi/CD133+ | |
| Melanoma | ABCB5+ | |

Examples of the cancer stem cells whose growth is intended to be inhibited in the present invention may include all stem cells of cancers as listed above, such as breast cancer, colorectal cancer, uterine cancer, fallopian tube cancer, ovarian cancer, gastric cancer, brain cancer, head and neck cancer, rectal cancer, small intestine cancer, esophageal cancer, lymph gland cancer, gallbladder cancer, lung cancer, skin cancer (or melanoma), kidney cancer, bladder cancer, blood cancer, pancreatic cancer, prostate cancer, thyroid cancer, endocrine gland cancer, oral cancer and liver cancer, with stem cells of breast cancer, colorectal cancer, uterine cancer, fallopian tube cancer, ovarian cancer, gastric cancer, brain cancer, head and neck cancer, rectal cancer, small intestine cancer, esophageal cancer, lymph gland cancer, gallbladder cancer, lung cancer, skin cancer (or melanoma), kidney cancer, bladder cancer, blood cancer, pancreatic cancer, prostate cancer, thyroid cancer, endocrine gland cancer, oral cancer, or liver cancer being preferred, and breast cancer stem cells, skin cancer stem cells, lung cancer stem cells or colorectal cancer stem cells being particularly preferred.

The above-mentioned cancer stem cells constantly undergo self-renewal, are capable of making tumors with less than a thousand cells in experimental animal models, and possess the capacity as malignant tumor cells. In addition, from the viewpoint that such cancer stem cells are surprisingly resistant to anti-cancer therapy and radiotherapy which are cancer therapies, elimination of such cancer stem cells is increasingly recognized as a barometer to determine success or failure of cancer therapy. Recently, it is recognized that even if cancer cells are killed using several conventional therapeutic methods such as surgery, radiotherapy, and anti-cancer chemotherapy, in a case where not all of cancer stem cells are killed, cancer may recur from remaining cancer stem cells. In order to prevent such recurrence of cancer, there is a growing interest in developing chemotherapies, which target cancer stem cells having the capacity to regenerate tumors, and treatment protocols intended to treat cancer based on the same.

It is suggested that stem cells in normal tissues regulate cell growth and differentiation by their self-renewal mechanism, whereas cancer stem cells are affected by tumor microenvironment factors surrounding tumor cells so that abnormal self-renewal and maintenance pathways are activated to achieve rapid accumulation, which allows the cells to become malignant and gain resistance to anti-cancer therapies, thereby ultimately causing recurrence of cancer. However, specific mechanism studies have yet to be conducted on true nature and interaction of tumor microenvironment factors that regulate accumulation and maintenance of cancer stem cells.

In the present invention, the “prevention” may include, without limitation, any act of blocking symptoms of cancer, or suppressing or delaying the symptoms, using the miRNA, expression vector, transformant, or pharmaceutical composition of the present invention.

In the present invention, the “treatment” may include, without limitation, any act of ameliorating or beneficially altering symptoms of cancer, using the miRNA, expression vector, transformant, or pharmaceutical composition of the present invention.

The miRNA, expression vector, transformant, or pharmaceutical composition of the present invention may be further co-administered with other anti-cancer agents, so that their growth inhibitory effect on cancer cells and cancer stem cells or their cancer metastasis inhibitory effect can be further enhanced.

Here, for the anti-cancer agent, at least one selected from the group consisting of, but not limited to, the following anti-cancer agents may be used: nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, bortezomib, sunitinib, carboplatin, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate chitosan, gemcitabine, doxifluridine, pemetrexed, tegafur, capecitabine, gimeracil, oteracil, azacitidine, methotrexate, uracil, cytarabine, fluorouracil, fludarabine, enocitabine, flutamide, decitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, peplomycin, temsirolimus, temozolomide, busulfan, ifosfamide, cyclophosphamide, melphalan, altretamine, dacarbazine, thiotepa, nimustine, chlorambucil, mitolactol, leucovorin, tretinoin, exemestane, aminogluthetimide, anagrelide, navelbine, fadrozole, tamoxifen, toremifene, testolactone, anastrozole, letrozole, vorozole, bicalutamide, lomustine and carmustine.

In the present invention, the miRNA, expression vector, transformant, or pharmaceutical composition may be characterized by being in the form of capsules, tablets, granules, injections, ointments, powders, or beverages, and the miRNA, expression vector, transformant, or pharmaceutical composition may be characterized by being targeted to humans.

The miRNA, expression vector, transformant, or pharmaceutical composition may be formulated in the form of, but not limited to, oral preparations such as powders, granules, capsules, tablets, and aqueous suspensions, preparations for external use, suppositories, and sterile injectable solutions, respectively, according to conventional methods, and used. The miRNA, expression vector, or transformant of the present invention may be administered together with a pharmaceutically acceptable carrier, and the pharmaceutical composition of the invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a fragrance, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the miRNA, expression vector, transformant, or pharmaceutical composition of the present invention may be prepared in various ways by being mixed with the pharmaceutically acceptable carrier as described above. For example, for oral administration, the miRNA, expression vector, transformant, or pharmaceutical composition may be formulated in the form of tablets, troches, capsules, elixirs, suspensions, syrups, wafers, or the like. For injections, the miRNA, expression vector, transformant, or pharmaceutical composition may be formulated in the form of unit dosage ampoules or multiple dosage forms. Alternatively, the miRNA, expression vector, transformant, or pharmaceutical composition may be formulated into solutions, suspensions, tablets, capsules, sustained-release preparations, or the like.

Meanwhile, as examples of carriers, excipients, or diluents suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the miRNA, expression vector, transformant, or pharmaceutical composition according to the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

In the present invention, the “parenteral” includes subcutaneous, intradermal, intravenous, intramuscular, intraarticular, intrabursal, intrasternal, intradural, intralesional, and intracranial injection or infusion techniques. The miRNA, expression vector, transformant, or pharmaceutical composition of the present invention may also be administered in the form of suppositories for rectal administration.

The miRNA, expression vector, transformant, or pharmaceutical composition of the present invention may vary depending on a variety of factors, including activity of a certain compound used, the patient's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the present invention in any way. The miRNA, expression vector, transformant, or pharmaceutical composition according to the present invention may be formulated in the form of pills, sugar-coated tablets, capsules, liquids, gels, syrups, slurries, or suspensions.

An expression vector comprising the miRNA of the present invention is specifically contained in an amount of 0.01 to 500 mg, and more specifically contained in an amount of 0.1 to 300 mg; and recombinant virus containing the miRNA of the present invention is specifically contained in an amount of 10³ to 10¹² IU (10 to 10¹⁰ PFU), and more specifically contained in an amount of 10⁵ to 10¹⁰ IU. However, the present invention is not limited thereto.

In addition, a transformant comprising the miRNA of the present invention is specifically contained in an amount of 10³ to 10⁸, and more specifically contained in an amount of 10⁴ to 10⁷. However, the present invention is not limited thereto.

In addition, an effective dose of a composition that comprises, as an active ingredient, an expression vector or transformant comprising the miRNA of the present invention is, based on kg body weight, 0.05 to 12.5 mg/kg for vectors, 10⁷ to 10¹¹ virus particles (10⁵ to 10⁹ IU)/kg for recombinant viruses, 10³ to 10⁶ cells/kg for cells, and specifically 0.1 to 10 mg/kg for vectors, 10⁸ to 10¹⁰ virus particles (10⁶ to 10⁸ IU) for recombinant viruses, and 10² to 10⁵ cells/kg for cells. Administration may be made 2 to 3 times a day. The composition as described above is not necessarily limited thereto, and may vary depending on the patient's condition and severity of disease.

### Advantageous Effects of Invention

The miRNA provided by the present invention can effectively inhibit growth of cancer cells as well as cancer stem cells so that cancer is prevented and/or treated; and, furthermore, the miRNA can also prevent drug resistance, metastasis, and recurrence of cancer.

### BRIEF DESCRIPTION OF DRAWINGS

FIG. 1A graphically illustrates results obtained by subjecting MCF7 breast cancer cells to treatment with the miRNA according to the present invention and then analyzing cell viability, in Example 1 of the present invention.

FIG. 1B graphically illustrates results obtained by subjecting BT474 breast cancer stem cells to treatment with the miRNA according to the present invention and then analyzing cell viability, in Example 1 of the present invention.

FIG. 2A graphically illustrates results obtained by subjecting BT474 breast cancer stem cells to treatment with the miRNA according to the present invention and then analyzing changes in relative colony number, in Example 2 of the present invention.

FIG. 2B graphically illustrates results obtained by subjecting HCT15 colorectal cancer stem cells to treatment with the miRNA according to the present invention and then analyzing changes in relative colony number, in Example 2 of the present invention.

FIG. 3A graphically illustrates results obtained by subjecting SK-MEL-28 melanoma cells to treatment with hsa-miR-6514-5p miRNA according to the present invention and then analyzing cell viability, in Example 3 of the present invention.

FIG. 3B graphically illustrates results obtained by subjecting SK-MEL-28 melanoma cells to treatment with hsa-miR-503-3p miRNA according to the present invention and then analyzing cell viability, in Example 3 of the present invention.

FIG. 3C graphically illustrates results obtained by subjecting SK-MEL-28 melanoma cells to treatment with the positive control, hsa-miR-34a, and then analyzing cell viability, in Example 3 of the present invention.

FIG. 4A graphically illustrates results obtained by subjecting NCI-H460 lung cancer cells to treatment with hsa-miR-6514-5p miRNA and then analyzing cell viability, in Example 4 of the present invention.

FIG. 4B graphically illustrates results obtained by subjecting NCI-H460 lung cancer cells to treatment with hsa-miR-503-3p miRNA and then analyzing cell viability, in Example 4 of the present invention.

FIG. 4C graphically illustrates results obtained by subjecting NCI-H460 lung cancer cells to treatment with the positive control, hsa-miR-34a, and then analyzing cell viability, in Example 4 of the present invention

FIG. 5A graphically illustrates results obtained by subjecting BT474 breast cancer stem cells to treatment with the miRNA according to the present invention and then analyzing changes in expression level of NANOG, a stemness-related gene, in Example 5 of the present invention.

FIG. 5B graphically illustrates results obtained by subjecting BT474 breast cancer stem cells to treatment with the miRNA according to the present invention and then analyzing changes in expression level of OCT4, a stemness-related gene, in Example 5 of the present invention.

FIG. 5C graphically illustrates results obtained by subjecting BT474 breast cancer stem cells to treatment with the miRNA according to the present invention and then analyzing changes in expression level of CK18, a stemness-related gene, in Example 5 of the present invention.

FIG. 6A graphically illustrates results obtained by subjecting HCT15 colorectal cancer stem cells to treatment with the miRNA according to the present invention and then analyzing changes in expression level of NANOG, a stemness-related gene, in Example 5 of the present invention.

FIG. 6B graphically illustrates results obtained by subjecting HCT15 colorectal cancer stem cells to treatment with the miRNA according to the present invention and then analyzing changes in expression level of OCT4, a stemness-related gene, in Example 5 of the present invention.

FIG. 6C graphically illustrates results obtained by subjecting HCT15 colorectal cancer stem cells to treatment with the miRNA according to the present invention and then analyzing changes in expression level of KRT20, a stemness-related gene, in Example 5 of the present invention.

FIG. 7A graphically illustrates results obtained by subjecting SK-MEL-28 melanoma stem cells to treatment with the miRNA according to the present invention and then analyzing changes in expression level of NANOG, a stemness-related gene, in Example 6 of the present invention.

FIG. 7B graphically illustrates results obtained by subjecting SK-MEL-28 melanoma stem cells to treatment with the miRNA according to the present invention and then analyzing changes in expression level of OCT4, a stemness-related gene, in Example 6 of the present invention.

FIG. 8 illustrates invaded cancer cell count obtained by subjecting SK-MEL-28 melanoma cells to treatment with the miRNA according to the present invention and then counting the invaded cancer cells which have passed through Matrigel and attached to the transwell bottom, in Example 7 of the present invention.

FIG. 9 illustrates photographs of invaded cancer cells obtained by subjecting SK-MEL-28 melanoma cells to treatment with the miRNA according to the present invention and then photographing the invaded cancer cells which have passed through Matrigel and attached to the transwell bottom, in Example 7 of the present invention.

FIG. 10 illustrates invaded cancer cell count obtained by subjecting NCI-H460 lung cancer cells to treatment with the miRNA according to the present invention and then counting the invaded cancer cells which have passed through Matrigel and attached to the transwell bottom, in Example 8 of the present invention.

FIG. 11 illustrates photographs of invaded cancer cells obtained by subjecting NCI-H460 lung cancer cells to treatment with the miRNA according to the present invention and then photographing the invaded cancer cells which have passed through Matrigel and attached to the transwell bottom, in Example 8 of the present invention.

### DETAILED DESCRIPTION OF INVENTION

According to an embodiment of the present invention, there is provided a pharmaceutical composition for preventing or treating cancer, comprising, as an active ingredient, miRNA comprising a seed sequence represented by the nucleotide sequence of any one of SEQ ID NOs: 1 to 3; an expression vector comprising the miRNA; or a transformant transformed with the expression vector.

According to another embodiment of the present invention, there is provided a pharmaceutical composition for inhibiting growth of cancer stem cells, comprising, as an active ingredient, miRNA comprising a seed sequence represented by the nucleotide sequence of any one of SEQ ID NOs: 1 to 3; an expression vector comprising the miRNA; or a transformant transformed with the expression vector.

According to yet another embodiment of the present invention, there is provided a pharmaceutical composition for preventing or treating metastasis of cancer, comprising, as an active ingredient, miRNA comprising a seed sequence represented by the nucleotide sequence of any one of SEQ ID NOs: 1 to 3; an expression vector comprising the miRNA; or a transformant transformed with the expression vector.

Hereinafter, the present invention will be described in more detail by way of examples. These examples are only for describing the present invention in more detail, and it will be apparent to those skilled in the art that according to the gist of the present invention, the scope of the present invention is not limited by these examples.

### EXAMPLES

### [Preparation Example 1] Preparation of miRNAs

Three miRNAs represented by the nucleotide sequences as shown in Table 2 below were synthesized.

TABLE 2

| Type of miRNA | Nucleotide sequence |
|---|---|
| hsa-miR-328-3p miRNA | CUGGCCCUCUCUGCCCUUCCGU |
| | (SEQ ID NO: 4) |
| hsa-miR-6514-5p miRNA | UAUGGAGUGGACUUUCAGCUGGC |
| | (SEQ ID NO: 5) |
| hsa-miR-503-3p miRNA | GGGGUAUUGUUUCCGCUGCCAGG |
| | (SEQ ID NO: 6) |

### [Preparation Example 2] Preparation of Cancer Cells and Cancer Stem Cells

MCF-7, BT474, HCT15, and SK-MEL-28 cancer cell lines were purchased from ATCC and cultured in DMEM/F12 medium supplemented with B27 supplement and growth factors (FGF and EGF, 20 ng/mL each) to induce cancer stem cells. Only cells expressing the cancer stem cell marker, CD44+, were selected and cultured for about 2 weeks.

In addition, NCI-H460 cancer cell line was prepared as other cancer cells.

### [Example 1] Viability Evaluation for Cancer Cells and Cancer Stem Cells

Using lipofectamine, the three miRNAs prepared in Preparation Example 1 were introduced respectively into the MCF-7 and BT474 breast cancer stem cells prepared in Preparation Example 2 for 48 hours, and then changes in cell viability were compared. The results are illustrated in FIGS. 1A and 1B. Here, no treatment was performed for the negative control, and the positive control was subjected to treatment with 5-fluorouracil (5-FU) (Sigma, USA).

As illustrated in FIGS. 1A and 1B, it was found that in a case where the breast cancer stem cells are subjected to treatment with the miRNA according to the present invention, viability of the breast cancer stem cells is remarkably decreased.

From these results, it can be seen that the miRNAs according to the present invention have effects of inhibiting growth and proliferation of cancer cells or cancer stem cells and inducing death thereof.

### [Example 2] Evaluation of Inhibited Proliferation of Cancer Cells and Cancer Stem Cells

In the same manner as in Example 1, the three miRNAs prepared in Preparation Example 1 were introduced respectively into the BT474 breast cancer stem cells and HCT15 colorectal cancer stem cells prepared in Preparation Example 2, and then changes in colony number. The results are illustrated in FIGS. 2A and 2B. Here, no treatment was performed for the negative control, and the positive control was subjected to treatment with 5-fluorouracil (5-FU) (Sigma, USA).

As illustrated in FIGS. 2A and 2B, it was found that in a case where the breast cancer stem cells and colorectal cancer stem cells are respectively subjected to treatment with the miRNA according to the present invention, colony number of the cancer stem cells is remarkably decreased.

From these results, it can be seen that the miRNAs according to the present invention have effects of inhibiting growth and proliferation of cancer cells or cancer stem cells and inducing death thereof.

### [Example 3] Evaluation of Viability of Cancer Cells

Using lipofectamine, the hRNA-miR-6514-5p miRNA and the hsa-miR-503-3p miRNA prepared in Preparation Example 1 were introduced respectively into the SK-MEL-28 melanoma cells prepared in Preparation Example 2 for 48 hours, and then changes in cell viability with treatment concentrations were compared. The results are illustrated in FIGS. 3A to 3C. IC₅₀ for the melanoma cells and final concentration for killing the entire melanoma cells were measured. The results are as shown in Table 3 below. Here, no treatment was performed for the negative control; and for the positive control, miR-34a, the only miRNA mimic-based therapeutic agent that had entered clinical phase I, was used.

TABLE 3

| Item | IC₅₀ (nM) | Final Conc. (nM) |
|---|---|---|
| miR34a | 1.715 | 1715 |
| hsa-miR-6514-5p | 0.6800 | 680 |
| hsa-miR-503-3p | 0.02280 | 22 |

As shown in FIG. 3A to 3C and Table 3, it was found that in a case where the melanoma cells are subjected to treatment with the miRNA according to the present invention, viability of the melanoma cells is remarkably decreased, in which the viability of the melanoma cells is remarkably inhibited at a lower concentration than a case of being treated with the positive control, miR-34a.

From these results, it can be seen that the miRNAs according to the present invention have effects of inhibiting growth and proliferation of cancer cells and inducing death thereof.

### [Example 4] Evaluation of Viability of Cancer Cells

Using lipofectamine, the hRNA-miR-6514-5p miRNA and the hsa-miR-503-3p miRNA prepared in Preparation Example 1 were introduced respectively into the NCI-H460 lung cancer cells prepared in Preparation Example 2 for 48 hours, and then changes in cell viability with treatment concentrations were compared. The results are illustrated in FIGS. 4A to 4C. IC₅₀ for the melanoma cells and final concentration for killing the entire melanoma cells were measured. The results are as shown in Table 4 below. Here, no treatment was performed for the negative control; and for the positive control, miR-34a was used.

TABLE 4

| Item | IC₅₀ (nM) | Final Conc. (nM) |
|---|---|---|
| miR34a | 0.01030 | 10 |
| hsa-miR-6514-5p | 0.01718 | 17 |
| hsa-miR-503-3p | 0.003044 | 3 |

As shown in FIG. 4A to 4C and Table 4, it was found that in a case where the lung cancer cells are subjected to treatment with the miRNA according to the present invention, viability of the lung cancer cells is remarkably decreased to a level equivalent to or higher than a case of being treated with the positive control, miR-34a.

From these results, it can be seen that the miRNAs according to the present invention have effects of inhibiting growth and proliferation of cancer cells and inducing death thereof.

### [Example 5] Evaluation of Inhibited Stemness of Cancer Stem Cells

In the same manner as in Example 1, the three miRNAs prepared in Preparation Example 1 were introduced respectively into the BT474 breast cancer stem cells and HCT15 colorectal cancer stem cells prepared in Preparation Example 2. Then, the cancer stem cells into which the miRNA had been introduced were harvested. RNAs of NANOG, OCT4, CK18, and KRT20 were isolated, and then cDNAs were synthesized therefrom. Using the primers as shown in Table 5 below, qPCR technique was used to digitize and quantify the cycle at which gene is amplified in real-time. The results are as illustrated in FIGS. 5A to 5C and FIGS. 6A to 6C. Here, no treatment was performed for the negative control, and the positive control was subjected to treatment with napabucasin (Abcam, USA).

TABLE 5

| Primer name | Primer sequence | |
|---|---|---|
| Nanog | Forward | CCCCAGCCTTTACTCTTCCTA |
| | Reverse | CCAGGTTGAATTGTTCCAGGTC |
| OCT4 | Forward | GTGTTCAGCCAAAAGACCATCT |
| | Reverse | GGCCTGCATGAGGGTTTCT |
| CK18 | Forward | TGAGACGTACAGTCCAGTCCTT |
| | Reverse | GCTCCATCTGTAGGGCGTAG |
| KRT20 | Forward | AGGAGACCAAGGCCCGTTA |
| | Reverse | ATCAGTTGGGCCTCCAGAGA |
| GAPDH | Forward | AATCCCATCACCATCTTCCA |
| | Reverse | TGGACTCCACGACGTACTCA |

As illustrated in FIGS. 5A to 5B and FIGS. 6A to 6C, it was found that in a case where the BT474 breast cancer stem cells and HCT15 colorectal cancer stem cells are subjected to treatment with the miRNA according to the present invention, expression levels of the stemness-related genes, NANOG and OCT14, are remarkably decreased. As illustrated in FIGS. 5C and 6C, it can be observed that expression levels of CK18 and KRT20 whose expression is inhibited in cancer stem cells are increased.

From these results, it can be seen that the miRNAs according to the present invention have an effect of causing cancer stem cells to lose stemness.

### [Example 6] Evaluation of Inhibited Stemness of Cancer Stem Cells

In the same manner as in Example 1, the hsa-miR-6514-5p miRNA and the hsa-miR-503-3p miRNA prepared in Preparation Example 1 were introduced respectively into the SK-MEL-28 melanoma stem cells prepared in Preparation Example 2. Then, the cancer stem cells into which the miRNA had been introduced were harvested. RNAs of NANOG and OCT4 were isolated, and then cDNAs were synthesized therefrom. Using the primers as shown in Table 5 of Example 5, qPCR technique was used to digitize and quantify the cycle at which gene is amplified in real-time. The results are illustrated in FIGS. 7A to 7B.

Here, no treatment was performed for the negative control; and for the positive control, miR-34a was used.

As illustrated in FIGS. 7A to 7B, it was found that in a case where the SK-MEL-28 melanoma stem cells are subjected to treatment with the miRNA according to the present invention, expression levels of the stemness-related genes, NANOG and OCT14, are remarkably decreased.

From these results, it can be seen that the miRNAs according to the present invention have an effect of causing cancer stem cells to lose stemness.

### [Example 7] Evaluation of Invasion Inhibition Capacity of Cancer Cells

The hsa-miR-6514-5p miRNA and the hsa-miR-503-3p miRNA prepared in Preparation Example 1 were introduced respectively into the SK-MEL-28 melanoma cells prepared in Preparation Example 2, and the resulting cells were incubated in transwells (Corning, USA) coated with 50 μl of 0.1× Matrigel (Corning, USA) for 2 days. Then, the invaded cancer cells, which had passed through the Matrigel and attached to the transwell bottom, were observed. For the respective cells, the results obtained by measuring the invaded cancer cell count and taking photographs of the cancer cells are illustrated in FIGS. 8 and 9. Here, no treatment was performed for the negative control; and for the positive control, miR-34a was used.

As illustrated in FIGS. 8 and 9, it was found that in a case where the melanoma cells are subjected to treatment with the miRNA according to the present invention, the invaded cell count is remarkably decreased as compared with the untreated negative control or a case of being treated with miR-34a. In particular, referring to FIG. 8, it was found that in a case where the melanoma cells are subjected to treatment with the miRNA according to the present invention, the invaded cell count is decreased by about 90% as compared with the negative control and is decreased by about 75% as compared with the positive control.

From these results, it can be seen that the miRNAs according to the present invention have an effect of inhibiting metastasis of cancer cells.

### [Example 8] Evaluation of Invasion Inhibition Capacity of Cancer Cells

The hsa-miR-6514-5p miRNA and the hsa-miR-503-3p miRNA prepared in Preparation Example 1 were introduced respectively into the NCI-H460 lung cancer cells prepared in Preparation Example 2, and the resulting cells were incubated in transwells (Corning, USA) coated with 50 μl of 0.1× Matrigel (Corning, USA) for 2 days. Then, the invaded cancer cells, which had passed through the Matrigel and attached to the transwell bottom, were observed. For the respective cells, the results obtained by measuring the invaded cancer cell count and taking photographs of the cancer cells are illustrated in FIGS. 10 and 11. Here, no treatment was performed for the negative control; and for the positive control, miR-34a was used.

As illustrated in FIGS. 10 and 11, it was found that in a case where the lung cancer cells are subjected to treatment with the miRNA according to the present invention, the invaded cell count is remarkably decreased as compared with the untreated negative control or a case of being treated with miR-34a. In particular, referring to FIG. 10, it was found that in a case where the lung cancer cells are subjected to treatment with the miRNA according to the present invention, the invaded cell count is decreased by about 60% as compared with the negative control and is decreased by about 40% as compared with the positive control.

From these results, it can be seen that the miRNAs according to the present invention have an effect of inhibiting metastasis of cancer cells.

### INDUSTRIAL APPLICABILITY

The present invention relates to a pharmaceutical composition capable of preventing, ameliorating, or treating cancer.

| Sequence List Free Text | |
|---|---|
| SEQ ID NO: 1: | UGGCCCU |
| SEQ ID NO: 2: | AUGGAGU |
| SEQ ID NO: 3: | GGGUAUU |
| SEQ ID NO: 4: | CUGGCCCUCUCUGCCCUUCCGU |
| SEQ ID NO: 5: | UAUGGAGUGGACUUUCAGCUGGC |
| SEQ ID NO: 6: | GGGGUAUUGUUUCCGCUGCCAGG |

1.-27. (canceled)

28. A method for preventing or treating cancer, the method comprising administering
miRNA as an active compound to a patient in need thereof, wherein the miRNA comprises a seed sequence represented by the nucleotide sequence of any one of SEQ ID NOs: 1 to 3;
an expression vector comprising the miRNA; or
a transformant transformed with the expression vector.

29. The method according to claim 28, wherein the miRNA comprises the nucleotide sequence represented by any one of SEQ ID NOs: 1 to 3 and consists of a consecutive nucleotide sequence having a total of 14 to 29 nucleotides.

30. The method according to claim 28, wherein the miRNA includes at least one of the following:
(1) at least one miRNA selected from the group consisting of hsa-miR-328-3p miRNA, hsa-miR-6514-5p miRNA, and hsa-miR-503-3p miRNA; and
(2) miRNA comprising the nucleotide sequence represented by any one of SEQ ID NOs: 1 to 3, the miRNA consisting of a nucleotide sequence that has at least 90% homology with the nucleotide sequence of (1).

31. The method according to claim 30, wherein the hsa-miR-328-3p miRNA consists of the nucleotide sequence of SEQ ID NO: 4.

32. The method according to claim 30, wherein the hsa-miR-6514-5p miRNA consists of the nucleotide sequence of SEQ ID NO: 5.

33. The method according to claim 30, wherein the hsa-miR-503-3p miRNA consists of the nucleotide sequence of SEQ ID NO: 6.

34. The method according to claim 28, wherein at least one of the nucleic acid molecules constituting the miRNA is in the following form:
a form that contains a phosphorothioate structure, the phosphorothioate structure being obtained by substitution of the phosphate backbone structure with elemental sulfur;
a form obtained by substitution with a DNA, peptide nucleic acid (PNA), or locked nucleic acid (LNA) molecule; or
a form obtained by substitution of the sugar 2′ hydroxyl group with a methylated, methoxylated, or fluorinated structure.

35. The method according to claim 28, wherein the miRNA is a miRNA precursor, primary miRNA (pri-miRNA), or a miRNA precursor in the form of a plasmid.

36. The method according to claim 28, wherein the miRNA is single-stranded or double-stranded.

37. The method according to claim 28, wherein the miRNA induces inhibited proliferation or death of cancer cells or cancer stem cells, inhibits stemness of cancer stem cells, or inhibits metastasis of the cancer cells or cancer stem cells.

38. The method according to claim 28, wherein the miRNA inhibits expression of at least one of NANOG and OCT4, or increases expression of at least one of CK18 and KRT20.

39. The method according to claim 28, wherein the cancer is selected from the group consisting of breast cancer, colorectal cancer, uterine cancer, fallopian tube cancer, ovarian cancer, gastric cancer, brain cancer, head and neck cancer, rectal cancer, small intestine cancer, esophageal cancer, lymph gland cancer, gallbladder cancer, lung cancer, skin cancer, kidney cancer, bladder cancer, blood cancer, pancreatic cancer, prostate cancer, thyroid cancer, endocrine gland cancer, oral cancer, and liver cancer.

40. The method according to claim 28, wherein the expression vector is a non-viral vector or a viral vector.

41. A method for inhibiting growth of cancer stem cells, the method comprising administering
miRNA as an active compound to a patient in need thereof, wherein the miRNA comprising a seed sequence represented by the nucleotide sequence of any one of SEQ ID NOs: 1 to 3;
an expression vector comprising the miRNA; or
a transformant transformed with the expression vector.

42. The method according to claim 41, wherein the miRNA contains the nucleotide sequence represented by any one of SEQ ID NOs: 1 to 3 and consists of a consecutive nucleotide sequence having a total of 14 to 29 nucleotides.

43. The method according to claim 41, wherein the miRNA includes at least one of the following:
(1) at least one miRNA selected from the group consisting of hsa-miR-328-3p miRNA, hsa-miR-6514-5p miRNA, and hsa-miR-503-3p miRNA; and
(2) miRNA containing the nucleotide sequence represented by any one of SEQ ID NOs: 1 to 3, the miRNA consisting of a nucleotide sequence that has at least 90% homology with the nucleotide sequence of (1).

44. The method according to claim 43, wherein the hsa-miR-328-3p miRNA consists of the nucleotide sequence of SEQ ID NO: 4.

45. The method according to claim 43, wherein the hsa-miR-6514-5p miRNA consists of the nucleotide sequence of SEQ ID NO: 5.

46. The method according to claim 43, wherein the hsa-miR-503-3p miRNA consists of the nucleotide sequence of SEQ ID NO: 6.

47. The method according to claim 41, wherein at least one of the nucleic acid molecules constituting the miRNA is in the following form:
a form that contains a phosphorothioate structure, the phosphorothioate structure being obtained by substitution of the phosphate backbone structure with elemental sulfur;
a form obtained by substitution with a DNA, peptide nucleic acid (PNA), or locked nucleic acid (LNA) molecule; or
a form obtained by substitution of the sugar 2′ hydroxyl group with a methylated, methoxylated, or fluorinated structure.

48. The method according to claim 41, wherein the miRNA is a miRNA precursor, primary miRNA (pri-miRNA), or a miRNA precursor in the form of plasmid.

49. The method according to claim 41, wherein the cancer is selected from the group consisting of breast cancer, colorectal cancer, uterine cancer, fallopian tube cancer, ovarian cancer, gastric cancer, brain cancer, head and neck cancer, rectal cancer, small intestine cancer, esophageal cancer, lymph gland cancer, gallbladder cancer, lung cancer, skin cancer, kidney cancer, bladder cancer, blood cancer, pancreatic cancer, prostate cancer, thyroid cancer, endocrine gland cancer, oral cancer, liver cancer, and blood cancer.

50. The method according to claim 41, wherein the expression vector is a non-viral vector or a viral vector.

51. A method for preventing or treating metastasis of cancer, the method comprising administering
miRNA as an active compound to a patient in need thereof, wherein the miRNA comprising a seed sequence represented by the nucleotide sequence of any one of SEQ ID NOs: 1 to 3;
an expression vector comprising the miRNA; or
a transformant transformed with the expression vector.


SEQUENCE LISTING (6 sequences)

SEQ ID NO: 1 - DNA, 7 bp, Artificial Sequence
  seed sequence of hsa-miR-328-3p miRNA
uggcccu

SEQ ID NO: 2 - DNA, 7 bp, Artificial Sequence
  seed sequence of hsa-miR-6514-5p miRNA
auggagu

SEQ ID NO: 3 - DNA, 7 bp, Artificial Sequence
  seed sequence of hsa-miR-503-3p miRNA
ggguauu

SEQ ID NO: 4 - DNA, 22 bp, Artificial Sequence
  hsa-miR-328-3p miRNA
cuggcccucucugcccuuccgu

SEQ ID NO: 5 - DNA, 23 bp, Artificial Sequence
  hsa-miR-6514-5p miRNA
uauggaguggacuuucagcuggc

SEQ ID NO: 6 - DNA, 23 bp, Artificial Sequence
  hsa-miR-503-3p miRNA
gggguauuguuuccgcugccagg